(19) United States
(12) Patent Application Publication
(10) Pub. No.: US 2004/0093007 A1
(43) Pub. Date: May 13, 2004

(54) Intraluminal occlusion device and method

(76) Inventors: Anthony Sussman, Savannah, GA (US); Christopher L. Wixon, Savannah, GA (US)

Correspondence Address: R. Anthony Diehl, 55 Nathan Cutler Drive, Bedford, NH, 03110, US

(21) Appl. No.: 10/445,716

(22) Filed: May 27, 2003

Related U.S. Application Data
(60) Provisional application No. 60/384,593, filed on May 31, 2002.
(60) Provisional application No. 60/396,488, filed on Jul. 17, 2002.

(51) Int. Cl. A61M 29/00

(52) U.S. Cl. 606/194; 606/158

(57) ABSTRACT

A vascular occlusion device for occluding a blood vessel including a lumen, a resilient deformable member in communication with the lumen, and a fluid-impervious cover surrounding the deformable member, wherein the deformable member has an expanded state at atmospheric pressure and a collapsed state at a second pressure that is lower than atmospheric pressure. Also, a method for occluding a blood vessel using a vascular occlusion device comprised of a lumen, a deformable member in communication with the lumen at the distal end of the lumen, and a fluid impervious cover surrounding the deformable member, including the steps of deflating the deformable member by reducing the pressure therein below atmospheric pressure, inserting the distal end into a blood vessel, and allowing the deformable member to inflate by allowing the pressure in the deformable member to return to atmospheric pressure.

### RELATED PATENT DOCUMENTS

This application claims priority to and is a conversion of U.S. Provisional Application Ser. No. 60/384,593, filed on May 31, 2002, entitled “Intraluminal Occlusion Device and Methods”, which is incorporated herein by reference in its entirety.

### FIELD OF THE INVENTION

The present invention relates to methods and devices for intraluminal occlusion, and, in particular to methods and devices for temporary vascular occlusion.

### BACKGROUND OF THE INVENTION

During peripheral vascular and cardiovascular surgical procedures, the construction of a surgical anastamosis requires a quiescent, bloodless surgical field. Accordingly, to prevent collateral blood flow from interfering with visualization of the surgical field, it generally is necessary to occlude both the proximal (inflow) and distal (outflow) portion of the vessel with either external or internal occlusion techniques.

The most common occlusion methods employ vascular clamps, silastic vessel loops or intravascular occluding devices. Unfortunately, known methods and corresponding devices tend to injure the underlying endothelial and smooth muscle cells within the blood vessel wall. Such techniques may damage the blood vessel wall and threaten graft patency, lead to stenosis or lead to other complications.

Of the currently available methods of occlusion, intravascular occluding devices have the lowest potential for blood vessel trauma. Unfortunately, currently available devices suffer from a number of deficiencies. For example, currently available intraluminal balloon catheters may be utilized for this purpose. However, the balloons on such balloon catheters are typically highly compliant, non-self limiting, and are typically manufactured from a smooth material with a low coefficient of friction so as to minimize “drag” along the vessel through which it is threaded. In addition, such balloon catheters typically are provided with a balloon that assumes a collapsed state at atmospheric pressure and may be inflated by increasing its internal pressure with air or other fluid. In addition, the shaft of the typical balloon catheter is constructed out of a material of sufficient rigidity to permit the passage of the catheter through the blood vessel and beyond the thrombus for which such catheters are generally designed to treat.

Accordingly, there are a number of disadvantages inherent in using the currently available balloon catheters for the purpose of occluding a blood vessel during construction of a vascular anastamosis. For example, because the balloon member is constructed from a smooth material with a low coefficient of friction, the balloon tends to “slide” from its previously placed intravascular position. In addition, the physical properties of such balloons cause the intraluminal pressure to be equally distributed in order to minimize wall tension of the balloon resulting in a generally spherical shape of the inflated balloon, causing the balloon to contact the vessel wall only at the equator, thereby decreasing the surface area in communication between the balloon and the vessel wall and increasing the tendency of the balloon to slide out of place. The operator may try to prevent this slippage by over-inflating the highly compliant balloon and delivering a greater force to the internal aspect of the blood vessel, thereby increasing the likelihood of damage to the vessel. The risk of over-inflation is increased because currently available balloon catheters typically must be actively inflated and further because such balloons are not self-limiting. As a result, the user must manually gauge the expansion of the balloon based on the perceived intraluminal pressure during hand inflation of the compliant balloon. Finally, Even when such catheters are successful at occluding the lumen, the shaft stiffness of currently available catheters impede the surgical field, making anastamotic suturing cumbersome.

One attempt to overcome these deficiencies is described in U.S. Pat. No. 4,946,463, which describes a device comprised of an occluding bulb constructed out of a hardened plastic material on the end of a catheter. Unfortunately, such a bulb must be precisely matched to the vessel diameter in order to properly occlude the vessel. Moreover, such devices fail to conform to irregularities in the shape of the vessel and, therefore, fail to reliably occlude the vessel. In addition, due to the rigidity of the material from which the occluder is constructed, there is potential for damage to the vessel during insertion and removal. Finally, because the catheter is static, there is a need to inventory numerous sizes.

Accordingly, there is a need for intravascular occlusion devices that effectively occlude a vessel during surgical anastamosis with minimal trauma to the vessel.

### SUMMARY OF THE INVENTION

The present invention is directed to a vascular occlusion device comprising a lumen, a resilient deformable member in communication with the lumen, and a fluid-impervious cover surrounding the deformable member. The deformable member has an expanded size at a first pressure and a collapsed size at a second pressure. The cover has a relatively high coefficient of friction. The first pressure is preferably atmospheric pressure. The device may further comprise a second lumen and a second deformable member. The device may further comprise a second lumen passing through the deformable member to serve as a shunt.

The present invention is also directed to a method for occluding a blood vessel using a vascular occlusion device comprised of a lumen, a deformable member in communication with the lumen at the distal end of the lumen, and a fluid impervious cover surrounding the deformable member, comprising the steps of: deflating the deformable member by reducing the pressure therein below atmospheric pressure, inserting the distal end into a blood vessel, and allowing the deformable member to inflate by allowing the pressure in the deformable member to return to atmospheric pressure.

### BRIEF DESCRIPTION OF THE DRAWINGS

These and other features and advantages of the present invention will be better understood by reading the following detailed description, taken together with the drawings wherein:

FIG. 1 is an elevational view of a vascular occlusion device in accordance with the present invention;

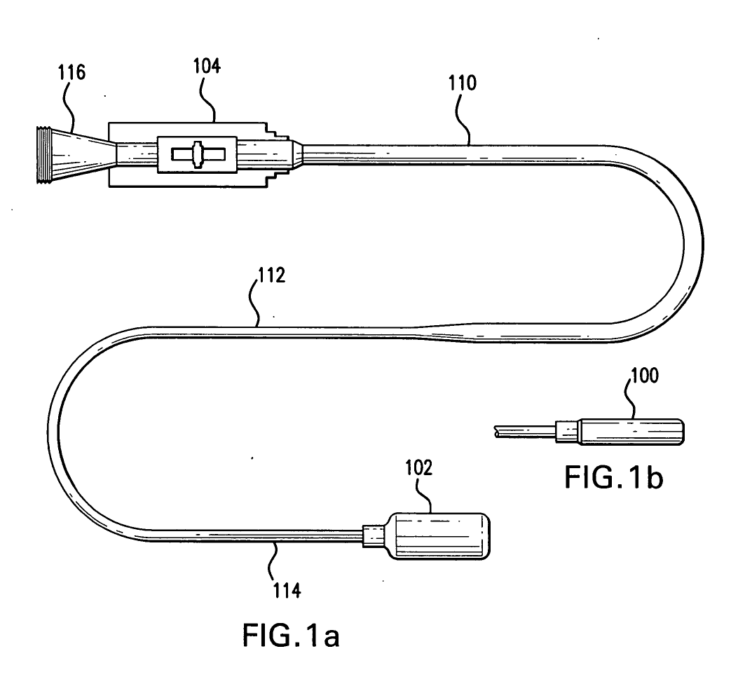

FIGS. 2a and 2b are schematic cross-sectional views of a vascular occlusion device in its collapsed and expanded state, respectively, in accordance with the present invention;

FIGS. 3a and 3b are schematic cross-sectional views of a vascular occlusion device in place in a vessel in its collapsed and expanded state, respectively, in accordance with the present invention; and

FIG. 4 is a chart of the compliance characteristics of various exemplary materials for use in an occlusion device in accordance with the present invention;

FIG. 5 is a schematic view of an alternative embodiment of an occlusion device in accordance with the present invention;

FIG. 6 is a schematic view of another alternative embodiment of an occlusion device in accordance with the present invention;

FIG. 7 is a schematic view of an alternative embodiment of a device in accordance with the present invention;

FIG. 8 is a schematic view of an alternative embodiment of the occlusion device used as an adjunct of a semi-rigid intravascular shunt;

FIG. 9 is a schematic view of an alternative embodiment of the occlusion device used as an adjunct of a semi-rigid intravascular shunt;

FIG. 10 is a schematic view of an alternative embodiment of the occlusion device used as an adjunct of a semi-rigid intravascular shunt;

FIG. 11 is a schematic view of an alternative embodiment of the occlusion device used as an adjunct of a semi-rigid intravascular shunt; and

FIG. 12 is a schematic view of an alternative embodiment of the occlusion device used as an adjunct of a semi-rigid intravascular shunt.

### DETAILED DESCRIPTION OF THE PREFERRED EMBODIMENTS

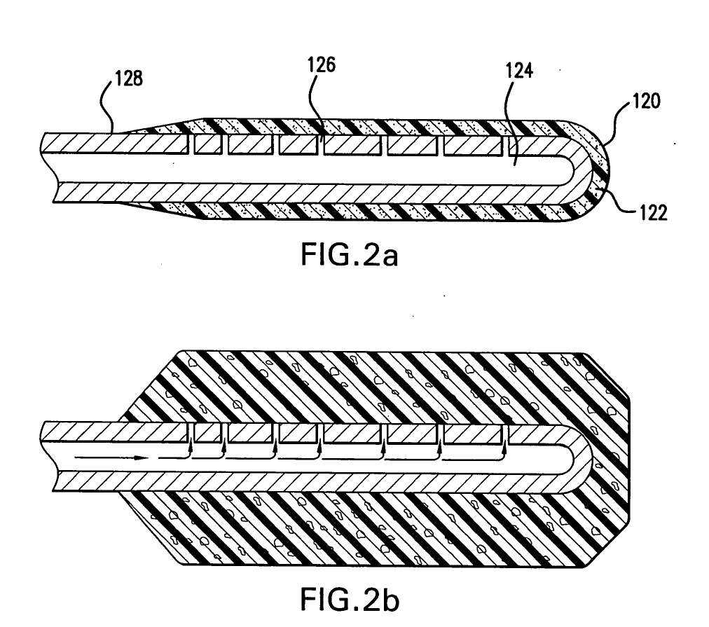

A vascular occlusion device 100 comprises a lumen 124, a resilient deformable member 122 in communication with the lumen and a fluid-impervious cover 120 surrounding the deformable member. The deformable member 122 has an expanded size at a first pressure (FIG. 2b) and a collapsed size at a second pressure (FIG. 2a). The cover 120 is constructed of a fluid impervious material, has a defined resting volume, is sufficiently compliant to allow expansion of the deformable member over a preferred pressure range, and has a coefficient of friction that minimizes migration of the deployed catheter from its desired location. The cover 120 and deformable member 122 are referred to herein collectively as the occlusion assembly.

The lumen 124 is preferably the bore of a single lumen catheter 112, though multiple lumen catheters may be used for additional or secondary functionality. The catheter 112 may be any catheter suitable for intravascular insertion and navigation, such as the catheters that are commonly employed in known balloon thrombectomy catheters. The catheter diameter may vary depending upon the size of the vessel to be occluded, but should generally have as small a diameter as possible (preferably, about 0.66 mm to about 1.33 mm) so long as it possesses sufficient structural integrity to be inserted into a vessel without occlusion of the lumen of the catheter. The catheter should also possess sufficient flexibility to move out of the operator's field while deployed. The distal end of the catheter 112 preferably has multiple openings 126 to permit the communication of air or other fluid with the deformable member 122, which preferably surrounds the distal end of the catheter.

The proximal end of the catheter preferably comprises means for forming a fluid-tight seal with a means for controllably changing the pressure in the catheter. For example, the proximal end of the catheter may have a standard Leur lock type of connection 116 that provides an airtight connection with any standard syringe, and serves as an deflation port. The proximal end of the catheter 112 also preferably comprises a valve mechanism 104 that allows the more distal portions of the catheter shaft to be either isolated, or open to pressures presented at the proximal end of the catheter. As used herein, the proximal end of the catheter is the end comprising the operator interface elements, such as the valve 104 and lock 116. The distal end of the catheter is the end comprising the occlusion assembly.

The deformable member 122 is preferably disposed around the circumference of the distal end of the catheter. The deformable member is preferably cylindrical in shape. The deformable member 122 may be formed from any suitable biocompatible, elastomeric material that is resilient in nature. Materials of different density may be utilized in order to achieve the desired function, which will be discussed in greater detail below. Suitable materials include foamed polyurethane or sponge-type silicone that may easily be compressed and restored. The deformable member is preferably bonded to the distal end of the catheter. In its non-deformed (expanded) condition, the deformable member is preferably substantially cylindrical. The outer circumference of the deformable member 122 in its deformed (collapsed) condition should be as small as possible. The outer circumference of the deformable member 122 in its expanded condition should be selected with regard to the size of the vessel to be occluded. The preferred compliance characteristics of the cover 120 are discussed below.

The cover 120, surrounds the deformable member 122. The cover 120 seals the distal end of the catheter 112 and, therefore, must be impervious to whatever fluid, preferably air, is used to expand and collapse the deformable member. The cover may also function as a constraint on the expansion of the deformable member. Finally, the cover 120 also engages the vessel wall and forms a substantially fluid-impervious seal within the lumen the vessel.

Accordingly, the cover 120 may be manufactured out of any type of highly elastic, biocompatible, polymeric material whose properties permit the expansion and collapsing of the elastomeric compound within the pressure ranges generated at the most proximal portion of the catheter. In addition, the cover 120 has a coefficient of friction that minimizes migration of the deployed catheter from its desired location. Preferably, the wet coefficient of friction of the expanded cover is greater than about 0.50. In addition, the cover may be coated with a biocompatible material, such as silica crystals, to further increase the coefficient of friction between the cover and the vessel wall. The thickness of the cover is preferably about 0.010 to about 0.1 mm in diameter when the deformable member 122 is in its expanded form. Specific diameter, resting volume and compliance characteristics of the cover are discussed in greater detail below. Suitable materials include, without limitation, latex, medical grade silicone rubbers, polyurethanes and the like.

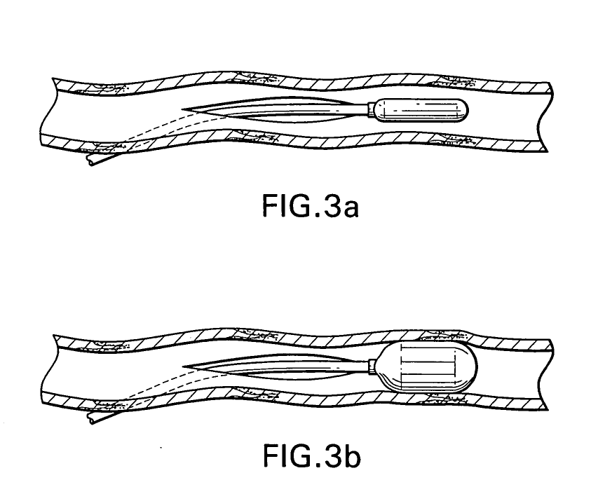

During deployment of the occlusion device, the valve 104 is opened and negative pressure is applied to the catheter via, for example, a syringe, which is attached to the proximal most portion of the catheter. The negative pressure is transmitted through the catheter shaft, to the deformable member 122 and causes the occlusion assembly to collapse (FIG. 3a). While negative pressure is applied to the end of the catheter, the flow switch (104) is, preferably, closed to prevent the balloon from expanding. After the occlusion assembly is directed into the desired position in the lumen of the vessel, the flow switch is opened to atmospheric pressures, thereby causing expansion of the deformable member. Notably, the expansion of the occlusion assembly is passive in that expansion occurs upon the release or withdrawal of the negative pressure and proceeds to an expanded condition without further manual inflation. In particular, the deformable member will stop expanding when it comes into contact with the vessel walls thereby occluding the vessel. The compliance characteristics of the deformable member 122 and the cover 120 are selected such that the occlusion assembly will put minimal pressure on the vessel walls in its expanded condition.

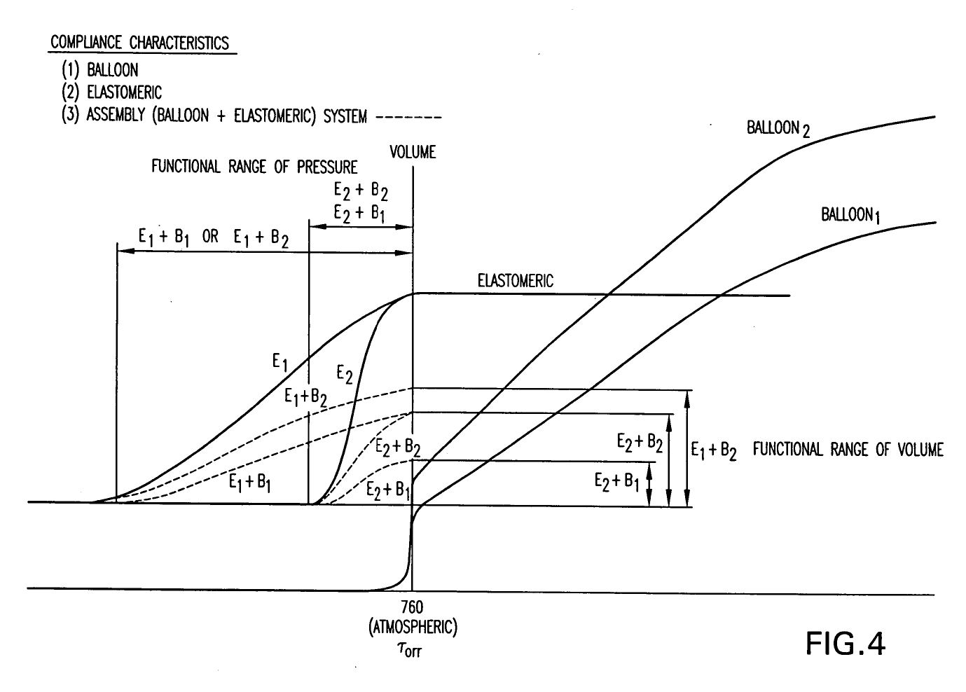

Accordingly, an important aspect of the invention is the selection and matching of the deformable member 122 and the cover 120. In particular, the performance of the occlusion device depends largely upon the combined elastic modulus of the deformable member 122 and the cover 120. FIG. 4 shows a chart of the compliance characteristics of two hypothetical covers 420a and 420b and two hypothetical deformable members 422a and 422b. Each of these elements could be combined to produce the assemblies 425A, 425B, 425C and 425D, each of which has unique combined elastic modulus.

Preferably, the catheters would be produced in a variety of distinct sizes each functioning over a range of blood vessel diameters. Table 1 lists exemplary dimensions of occlusion devices in accordance with the present invention. In Table 1 resting cover volume is the independent volume of the cover at atmospheric pressure (1 atm) and the expanded cover volume is the volume of the cover/expandable member assembly at atmospheric pressure (1 atm).

TABLE 1

| | | | | | Blood | | | |
|---|---|---|---|---|---|---|---|---|
| | | | | | vessel | Suitable | Resting | |
| | Shaft | Collapsed | Expanded | Assembly | functional | blood | cover | Expanded |
| | size | diameter | diameter | length | size | vessel | volume | cover volume |
| small | 2 F | 1 mm | 3.5 mm | 15 mm | 1.5-3.0 | Pedal, | 11.7 mm³ | 144.3 mm³ |
| | | | | | mm | tibial, | | |
| | | | | | | coronary | | |
| medium | 3 F | 2 mm | 6 mm | 20 mm | 2.5-5.0 | Brachial, | 62.8 mm³ | 565 mm³ |
| | | | | | mm | popliteal | | |
| large | 4 F | 3 mm | 10 mm | 25 mm | 4.0-8.0 | Femoral, | 176.7 mm³ | 7853 mm³ |
| | | | | | mm | iliac, | | |
| | | | | | | sublcavian | | |

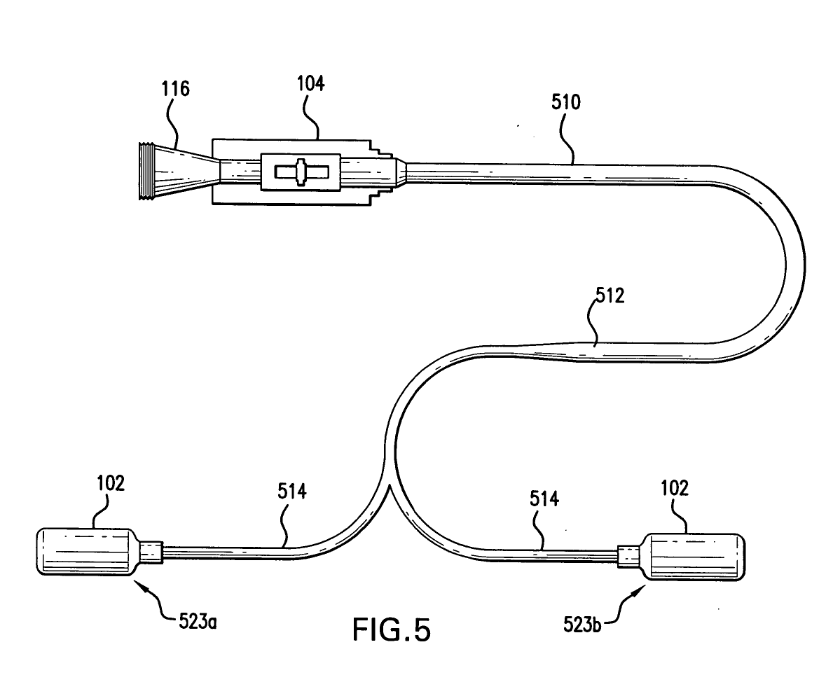

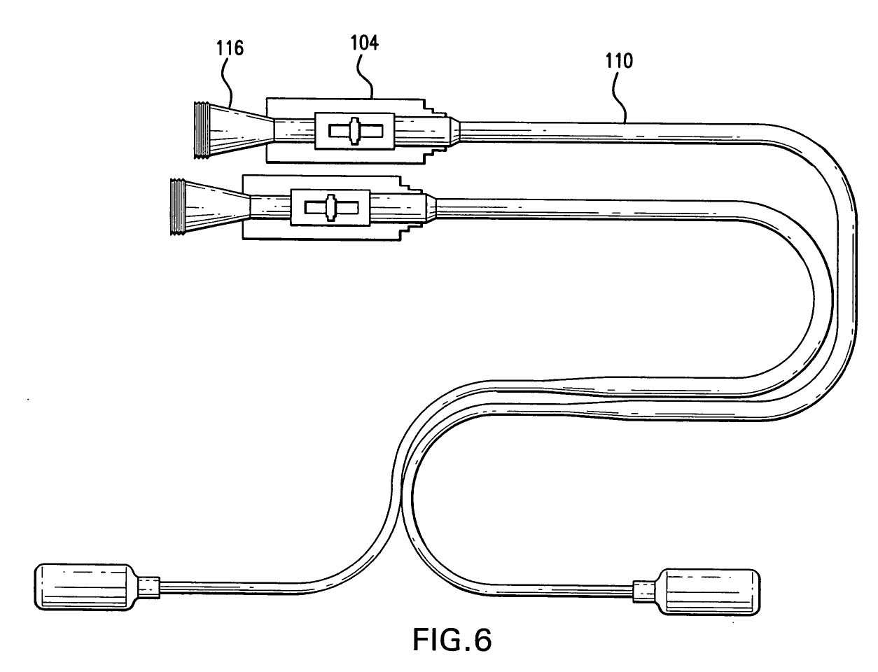

Referring now to FIG. 5, there is shown an alternative embodiment of the present invention comprising a pair of occlusion assemblies 523a and 523b at each distal end of a bifurcated catheter shaft 510. The bifurcated catheter may be formed from a proximal portion 512 coupled to a distal portion 514 in a T-shaped or Y-shaped (not shown) arrangement. Alternatively, the bifurcated catheter may be formed from 2 separate catheters running from the valve to the occlusion assemblies in a V-shaped arrangement, as shown in FIG. 6. In either case, the dual-occlusion assemblies are employed in the same manner as the single occlusion device to simultaneously occlude both ends of a vessel during anastamosis.

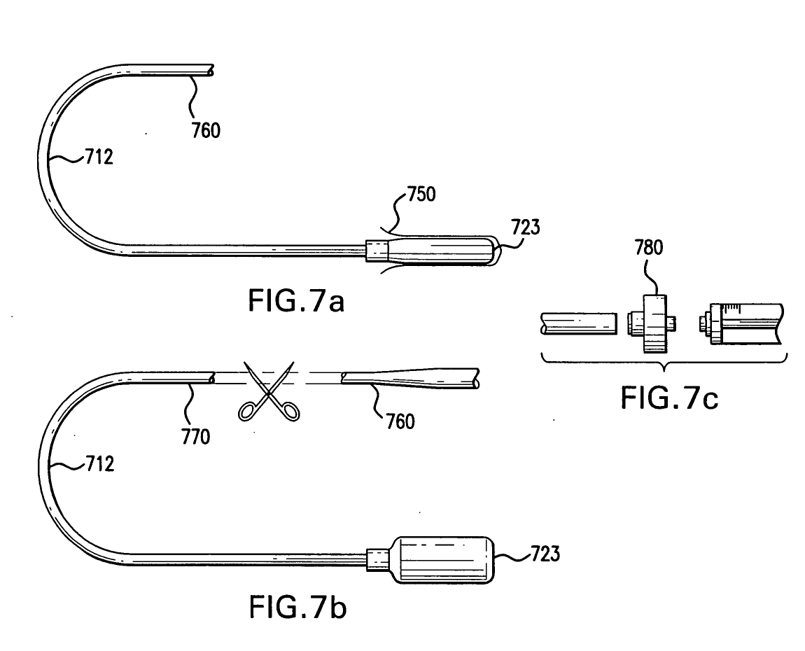

FIG. 7 shows a schematic view of an alternative embodiment of the occlusion device. The balloon assembly 723 is maintained in its collapsed form by a rigid constraining sheath 750. Additionally, the proximal portion of the shaft 712 is sealed 760 in such a manner to prevent air or fluid communication with the catheter shaft 712 and balloon assembly 723. The catheter is deployed by removing the restraining sheath 750, placing the balloon assembly 723 into its desired intravascular position, and cutting the proximal end of the catheter 770 with an ordinary pair of surgical scissors. Atmospheric pressure is then transmitted to the balloon assembly 723 via the catheter shaft 712, permitting expansion of the elastomeric compound to seek out its expanded form. When the function of intravascular occlusion is no longer required, the balloon assembly is converted back to its collapsed form by attaching, for example, a Touy-Borst Leur lock mechanism 780 to the catheter shaft and applying negative pressure to the catheter shaft and the balloon assembly using an ordinary syringe.

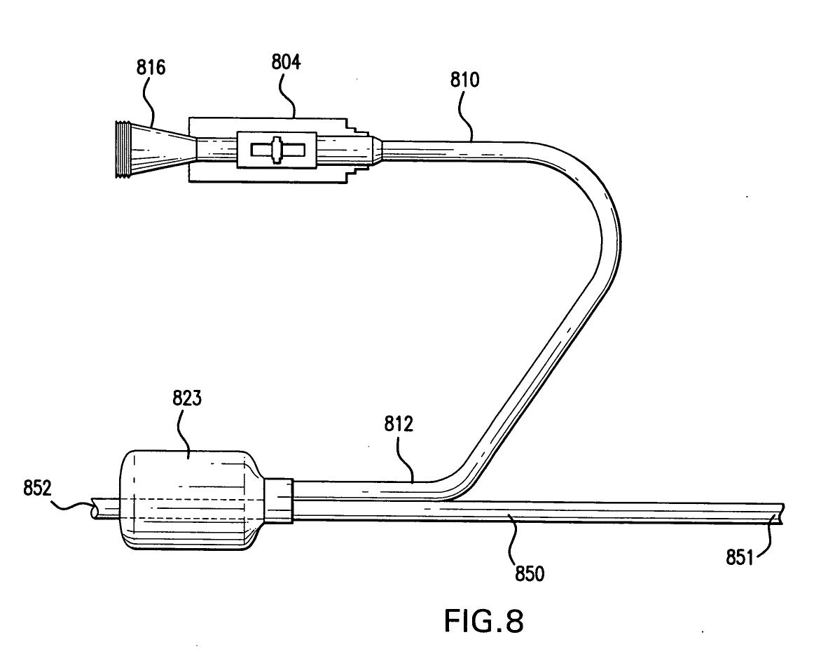

FIG. 8 shows an alternative embodiment of the device in that the balloon assembly 823 is disposed around a flexible intravascular shunt 850. The shunt may be constructed from any resilient material having a lumen to permit the passage of blood from a proximal 851 to a distal end 852. The tube may be constructed from silicone, silastic or other hardened plastic material. The occlusion assembly is circumferentially bonded to the distal end of the intravascular shunt and the distal portion of the catheter shaft 812 remains bonded to the intravascular shunt before departing in a T-shaped pr Y-shaped pattern.

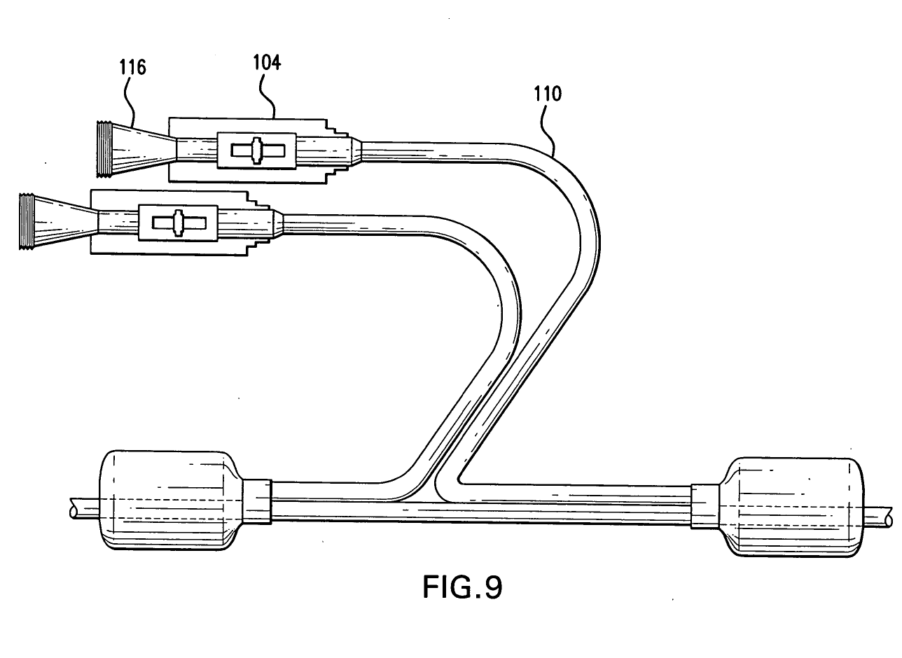

FIG. 9 shows a similar device to that shown in FIG. 8 with independently functioning balloon assembly units disposed around both the proximal and distal end of the intravascular shunt.

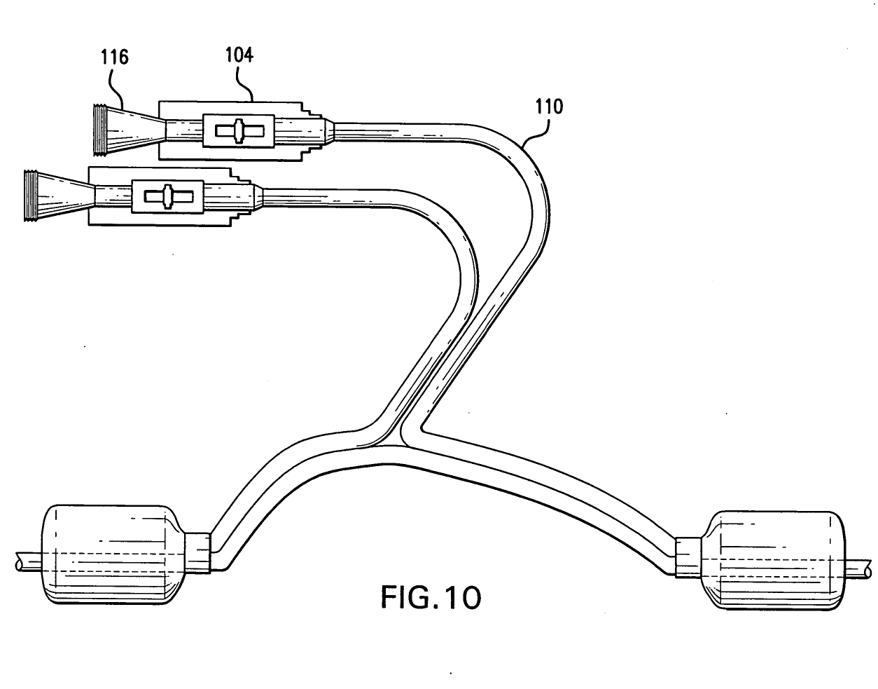

FIG. 10 shows a similar device to that shown in FIG. 9 in which the intravascular shunt is elongated and flexible to facilitate clinical application of the device.

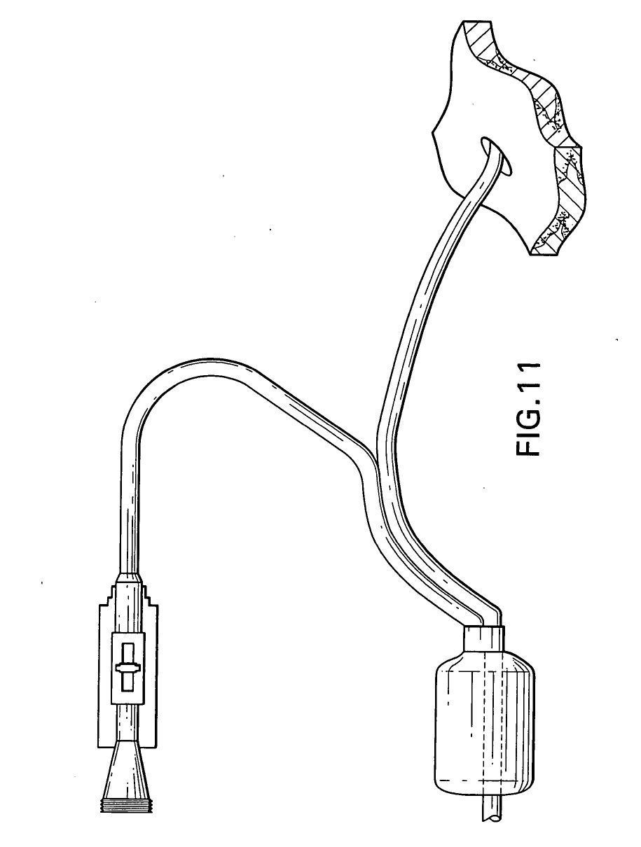

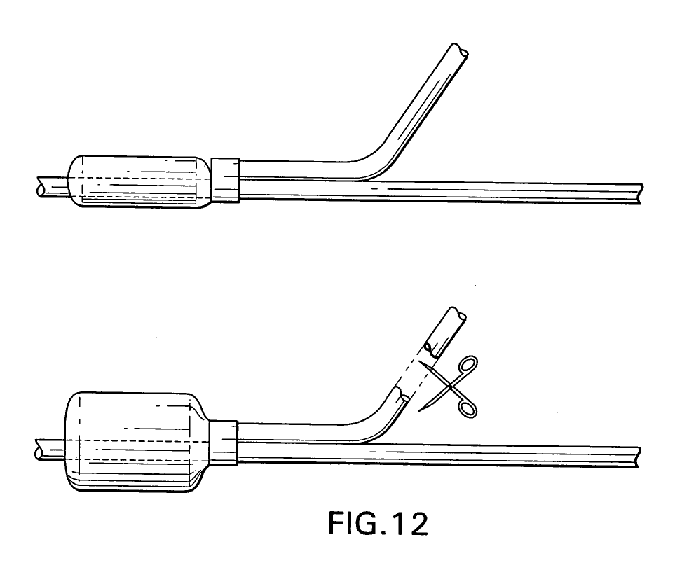

FIGS. 11 and 12 show combinations of alternative embodiments of portions of the present invention. A wide range of balloon sizes, shapes and lengths are possible which may be coupled with a variety of diameter intravascular shunts in order to suite the desired function of the catheter. Potential applications include, but are not limited to temporary shunting of the coronary artery, the carotid artery, femoral artery, or tibial artery.

Although the invention has been described herein in its preferred form, those of skill in the art will recognize that many modifications and changes may be made thereto without departing from the spirit and scope of the invention as set forth in the following claims.

What is claimed is:

1. A vascular occlusion device for occluding a blood vessel comprising:
a lumen,
a resilient deformable member in communication with the lumen, and
a fluid-impervious cover surrounding the deformable member, wherein
the deformable member has an expanded state at atmospheric pressure and a collapsed state at a second pressure that is lower than atmospheric pressure.

2. The vascular occlusion device of claim 1, further comprising a pressure control device for controlling the pressure in the lumen and the deformable member.

3. The vascular occlusion device of claim 1, wherein the cover has an outer surface having a relatively high coefficient of friction such that when the deformable member is placed in a blood vessel and is expanded to its expanded state, the device is held securely in the blood vessel without applying sufficient pressure to the walls of the blood vessel to cause significant damage to the blood vessel.

4. The vascular occlusion device of claim 1 further comprising a second lumen and a second deformable member in communication with the second lumen.

5. The vascular occlusion device of claim 4, further comprising a second pressure control device for controlling the pressure in the second lumen and the second deformable member.

6. The vascular occlusion device of claim 1 further comprising a second lumen passing through the deformable member to serve as a shunt through the deformable member.

7. The vascular occlusion device of claim 2, further comprising
a second lumen and a second deformable member in communication with the second lumen;
a second pressure control device for controlling the pressure in the second lumen and the second deformable member
a third lumen passing through the deformable member to serve as a shunt through the first deformable member;
a fourth lumen passing through the deformable member to serve as a shunt through the second deformable member,
wherein the cover has an outer surface having a relatively high coefficient of friction such that when the deformable member is placed in a blood vessel and is expanded to its expanded state, the device is held securely in the blood vessel without applying sufficient pressure to the walls of the blood vessel to cause significant damage to the blood vessel.

8. A method for occluding a blood vessel using a vascular occlusion device comprised of a lumen, a deformable member in communication with the lumen at the distal end of the lumen, and a fluid impervious cover surrounding the deformable member, comprising the steps of:
deflating the deformable member by reducing the pressure therein below atmospheric pressure,
inserting the distal end into a blood vessel, and allowing the deformable member to inflate by allowing the pressure in the deformable member to return to atmospheric pressure.
